(19) United States
(12) Patent Application Publication
(10) Pub. No.: US 2019/0070426 A1
(43) Pub. Date: Mar. 7, 2019

(54) IMPLANTED MAGNETIC SCREW ASSEMBLY FOR VERTEBRAL SPINE AND FRACTURED BONES

(71) Applicant: Imran Khurshid Alam, Plano, TX (US)

(72) Inventor: Imran Khurshid Alam, Plano, TX (US)

(21) Appl. No.: 16/122,005

(22) Filed: Sep. 5, 2018

Related U.S. Application Data
(60) Provisional application No. 62/555,603, filed on Sep. 7, 2017.

(51) Int. Cl. A61N 2/00; A61N 2/02; A61B 17/86

(52) U.S. Cl. CPC A61N 2/004; A61N 2/02; A61B 2017/00876; A61B 17/8605; A61B 17/864

(57) ABSTRACT

This technique will enhance the healing and stability of orthopedic bone implants to enhance bony growth around an implant including all skeletal bones, vertebral bodies, spinal pedicles, skull bone, dental implants, artificial joints and other skeletal fractures by inducing magnetic energy to the target tissue via a permanent magnet in the implant to enhance growth and apposition of the surrounding bone and tissue. In the preferred embodiment the implant holds a rigidly fixed, permanent magnet or magnets applying magnetic field of controlled frequency and intensity.

### BACKGROUND OF THE INVENTION

### Technical Field

The present invention relates to a magnetic screw assembly comprising a magnetic screw configured to traverse a patient's fractured bone or spinal vertebra or skeletal bone to stimulate the patient's bone growth (Osteogenesis) with a static magnetic field, and methods of use.

### Description of the Related Art

The Food and Drug Administration (FDA) has approved the magnetic devices for the treatment of nonunion and damaged bones that haven't healed over time. This can be the result of infection, poor blood supply or inadequate immobilization during the healing process. People with diabetes or osteoporosis, smokers and alcoholics are at elevated risk for poor bone healing.

Magnetic stimulation has been suggested as a possible therapeutic tool in treating the fractured bone healing and to enhance bone and spinal fusion in case of spinal instrumentation and neural structures. The studies suggest that exposure to a static magnetic field increases the rate of cutaneous wound healing by secondary intention and provide further testimony to the notion that magnetic fields can influence the physiology of the human body.

In view of the forgoing, one objective of the present invention is to provide an implantable magnet screw assembly for stimulating a patient's bone and skeleton with a static magnetic field.

### BRIEF SUMMARY OF THE INVENTION

According to a first aspect, the present disclosure relates to an implantable magnet screw assembly comprising a rod-shaped magnet housed within a screw. The screw has a head end and a point end and is configured to traverse a patient's bone with the point end located at the interior surface of the bone and the head end located at the exterior or interior surface of the bone. The implantable magnet assembly with the casing housing at least one flat magnet and arranged substantially perpendicularly with the rod-shaped magnet.

This magnetic screw will create a multi-lateral magnetic field in a desired region and will be placed strategically into the bone about the targeted areas, but not penetrating the spinal cavity. This screw consists of a hollow titanium alloy shaft which runs through the middle of the insulator assembly where magnet is placed. The screw will have a lower pullout-strength in comparison to a conventional screw.

The dimensions of magnetic screw for fractured bones, peripheral bones, vertebral bodies including laminar and pedicle application for spinal surgeries will be larger size depending on the applicable anatomic site.

In one embodiment, the implantable magnet assembly emits a magnetic field with a magnetic field strength of 2 mT-15 T as measured on an exterior surface.

In one embodiment, the implantable magnet assembly has one flat magnet which is cylindrically-shaped and aligned concentrically with the rod-shaped magnet with like magnetic poles of both magnets facing the same direction.

In one embodiment, the implantable magnet assembly has at least two flat magnets with edges arranged side-by-side in the casing.

In a further embodiment, where the implantable magnet assembly has at least two flat magnets with edges arranged side-by-side in the casing, not every flat magnet is arranged with magnetic poles facing in the same direction.

In another embodiment, where the implantable magnet assembly has at least two flat magnets with edges arranged side-by-side in the casing, all of the flat magnets are arranged with their magnetic poles in the same direction as the magnetic pole of the rod-shaped magnet.

In another embodiment, the exterior of the bone screw of the implantable magnet assembly is at least one of titanium, titanium alloy, stainless steel, cobalt alloy, magnetite, ferrite alloy, neodymium alloy, samarium alloy, Alnico, carbon fiber, polyethylene, polymethylmethacrylate, polyether ether ketone, or polycarbonate.

In another embodiment, the exterior surface of the casing comprises a tab portion, a notch, or a textured surface to facilitate a finger grip.

In another embodiment, the implantable magnet assembly has a fastening mechanism which may be a bayonet mount, a threaded connector, a clutch, a latch, a key and keyhole, a tongue and groove joint, a snap fastener, an R-clip, or a clamp, which is configured to removably attach the casing to the head end of the magnetic screw.

In another embodiment, the implantable magnet assembly also has a strap with a recess to receive the casing, wherein the strap secures the casing in place when encircling a part of the patient's anatomy.

According to a second aspect, the present disclosure relates to a method of delivering a static magnetic field to a patient's fractured bone or spinal vertebra or skeletal bone by implanting the implantable magnet assembly into the patient's fractured bone or spinal vertebra or skeletal bone wherein the magnetic screw traverses these areas with the point end located at the interior surface and the head end located at the exterior surface.

According to a third aspect, the present disclosure relates to a method of delivering a static magnetic field to a fractured bone or spinal vertebra or skeletal bone of a patient which involves implanting an implantable magnet into the affected area. This implantable magnet is a rod-shaped magnet housed within a magnetic screw, with it having a head end and a point end. When implanted, the screw traverses the patient's fractured bone, spinal vertebra or skeletal bone with the point end located at the interior surface and the head end located at the exterior surface.

In one embodiment of the method, the patient has osteoporosis or other bone disease, the ailment of the patient is treated.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a cross-sectional view of a multi-lateral magnetic field coming out of a magnetic screw

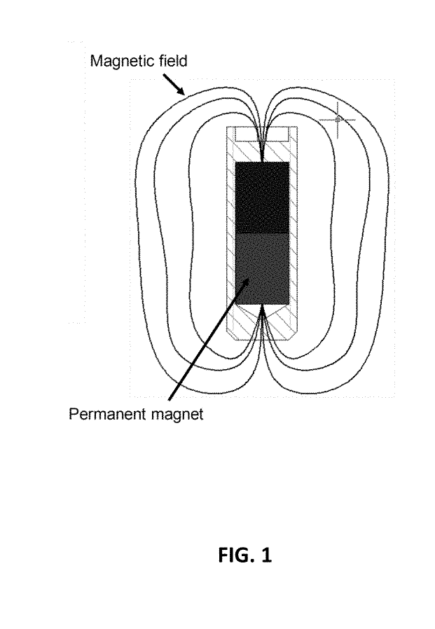

FIG. 2 is an example of a magnet-containing bone screw.

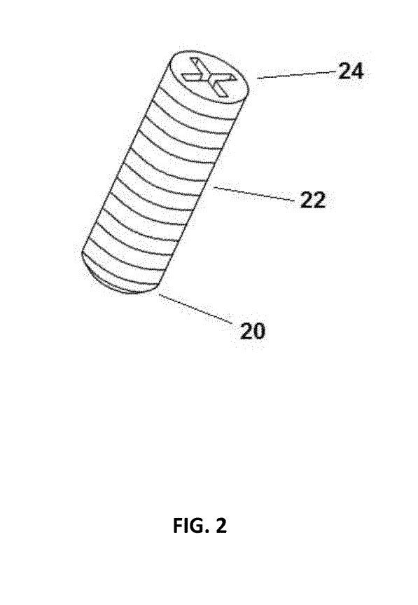

FIG. 3 is an exploded view of an implantable magnet assembly

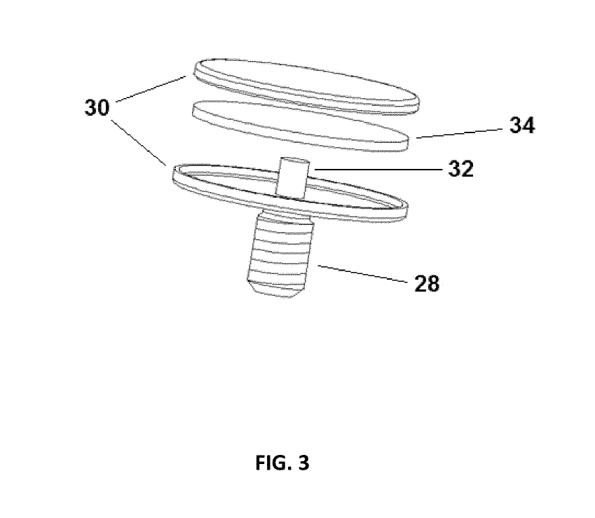

FIG. 4 is a cross-section view of the implantable magnet assembly of FIG. 3 showing the magnetic field lines.

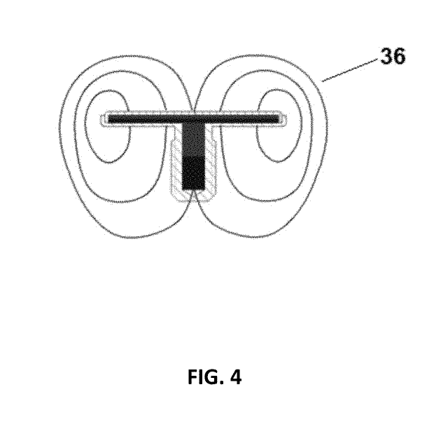

FIG. 5 is an example of an implantable magnet assembly with an indentation in a central portion of the casing.

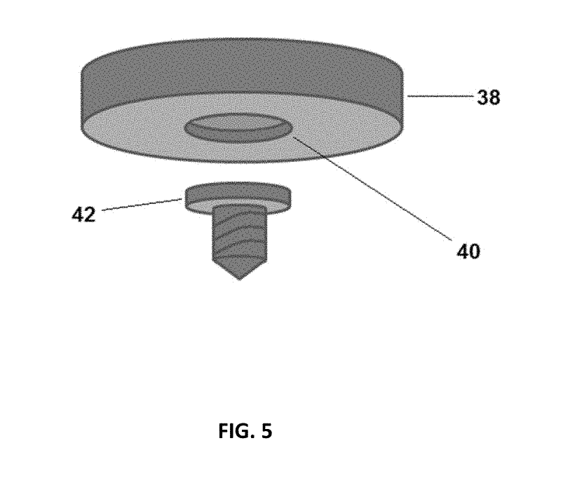

FIG. 6 illustrates and arrangement of the assembly of a magnetic screw for medical application: A blank metal has a hole drilled and a magnet is inserted, welded in place and machined to the screw to its final shape

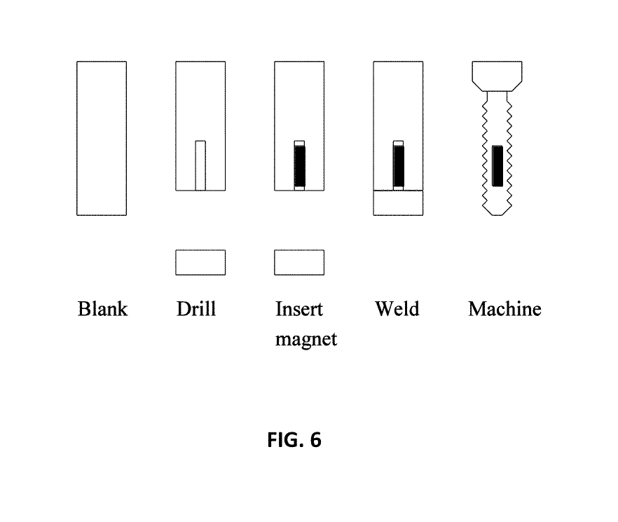

FIG. 7 shows the bone stimulation screw b-field plot representation of the field structure. The magnetic material here is NdFEB, grade 32 materials and the diagram shows the magnetic field for the screw. The vertical axis of the symmetry runs along the left side of the plot. The calculations are arranged so that they should be valid in 3D, so the magnets are truly cylinders. The semi-circular boundary is carefully arranged to behave as if it is in free space so as not to influence the field intensity by numerical artifacts.

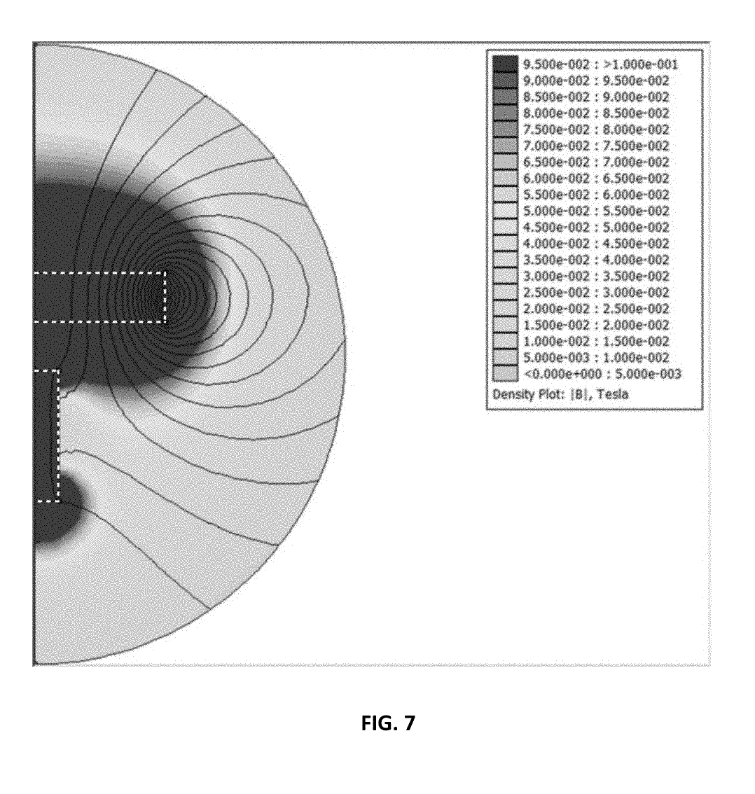

FIG. 8 is a diagrammatic view of differently designed screws (a) cancellous bone screw (b) cortical screw (c) illustrates an arrangement of spinal screws in different shapes and forms

FIG. 9 illustrates the screw assembly connected to a rod in order to stabilize the construct to facilitate bone fusion.

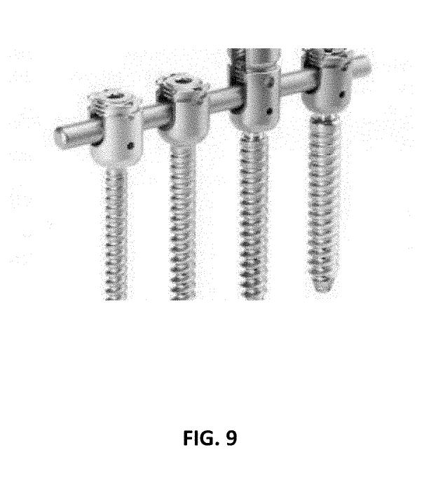

FIG. 10 shows differently designed screws for bone instrumentation

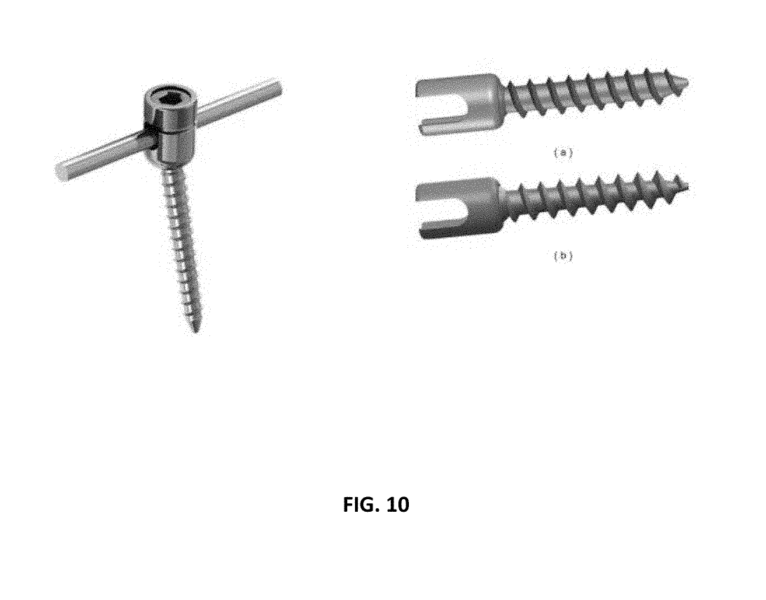

FIG. 11. Is a diagrammatic description of spinal screws placed within the cervical spinal cord, mid-thoracic spinal cord and lumbar spinal cord

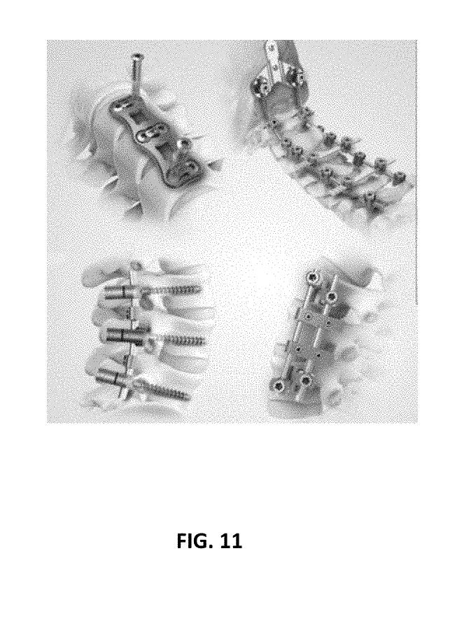

FIG. 12 shows a model for ileosacral spinal screw instrumentation

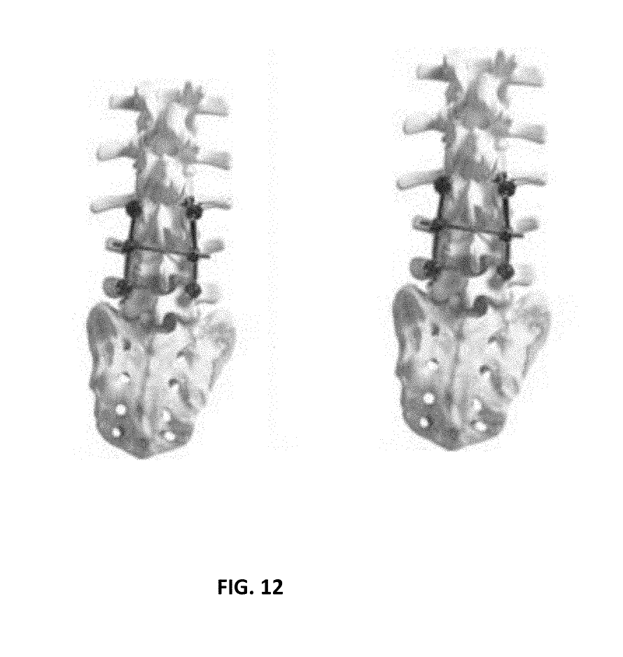

FIG. 13 shows segmental spinal instrumentation and rod fixation with different screws

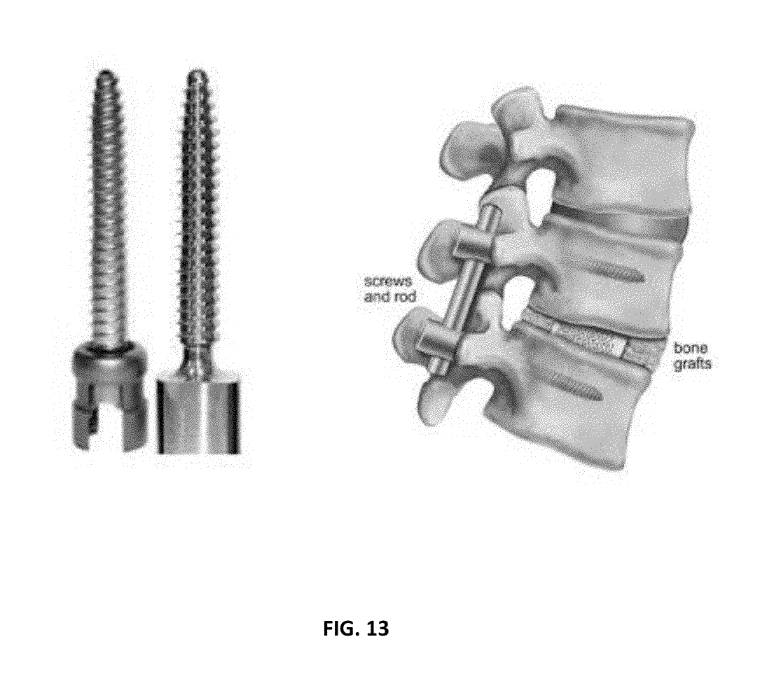

FIG. 14 illustrates an assortment of multi-shapes, sizes and constructs magnetic screws to be placed from scalp, bone, fractured bone, spinal bone and dental implants

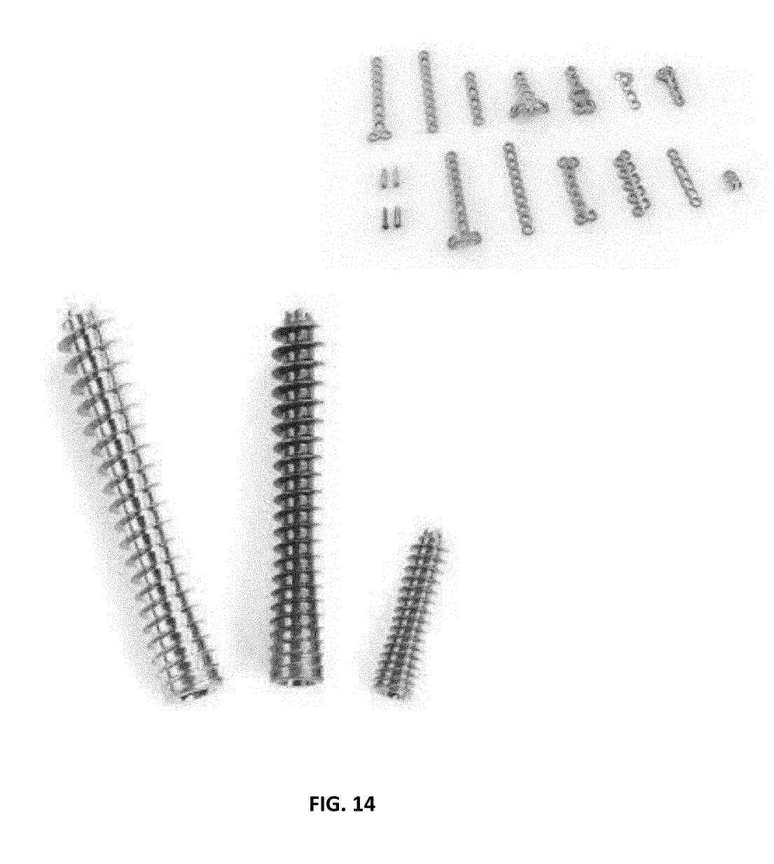

FIG. 15 is a cross sectional presentation of internal and external fixation of fractured bones with multiple magnetic screws to promote bone healing and fractures

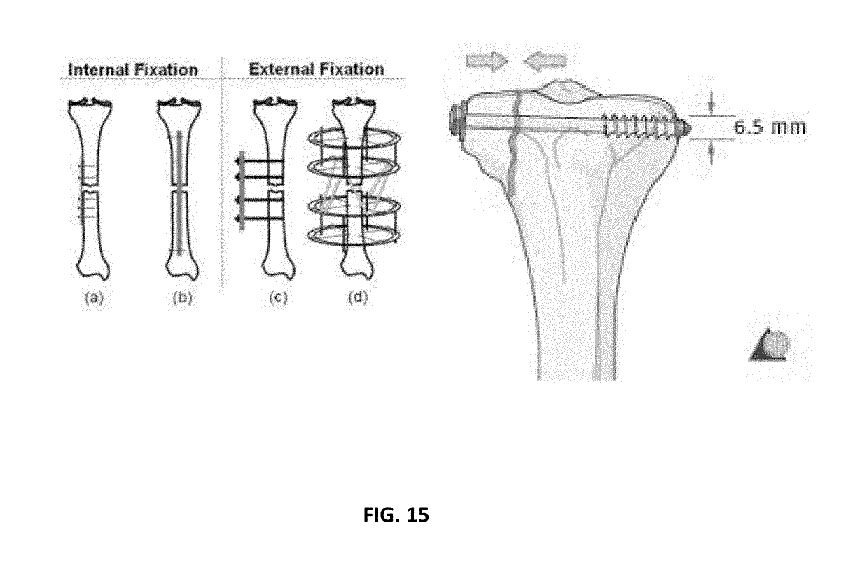

FIG. 16 shows a cross sectional multi-dimentaional view of (a) a fractured head of femur (b) fractured head and body of femur bone with magnetic screw for bone healing and stabilization

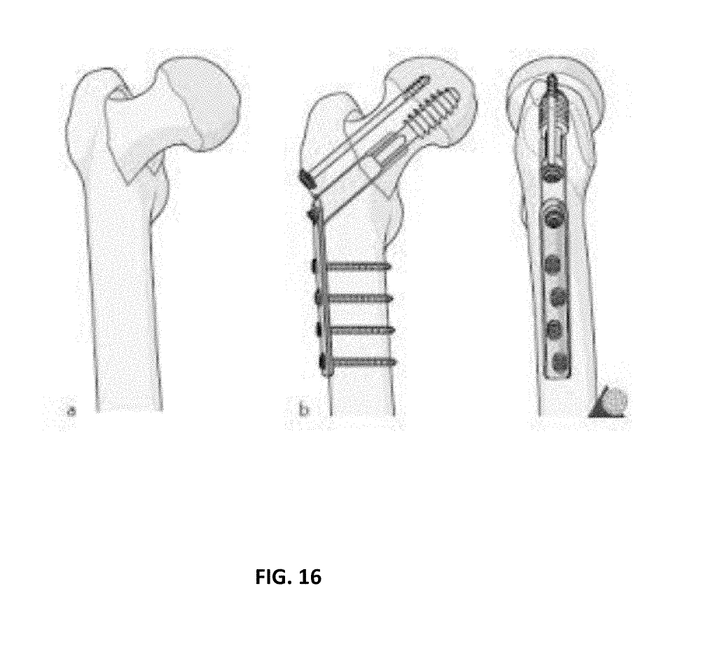

FIG. 17 is an illustration of specially designed screws for difficult fracture stabilization

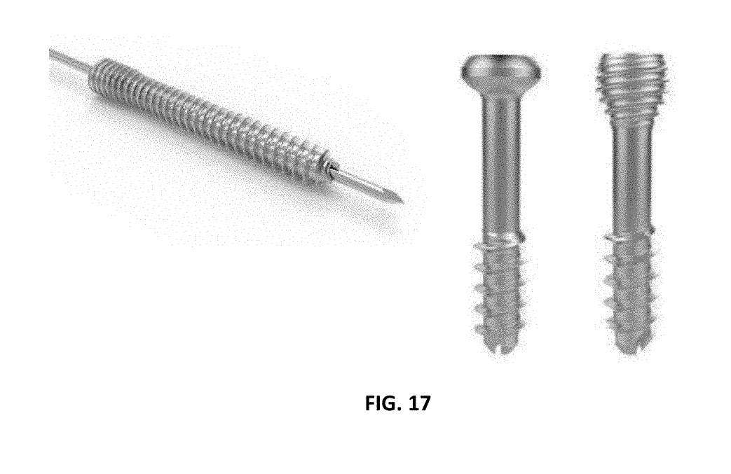

FIG. 18 a cross-sectional view of multiple trajectories used with magnetic screws stabilization and healing of bone fractures

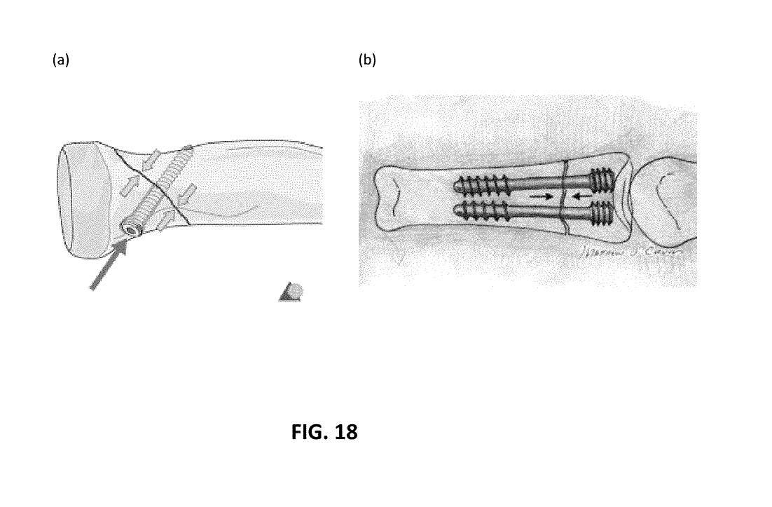

FIG. 19 illustrates clavicular instrumentation and fixation to stabilize the fracture with the magnetic screws

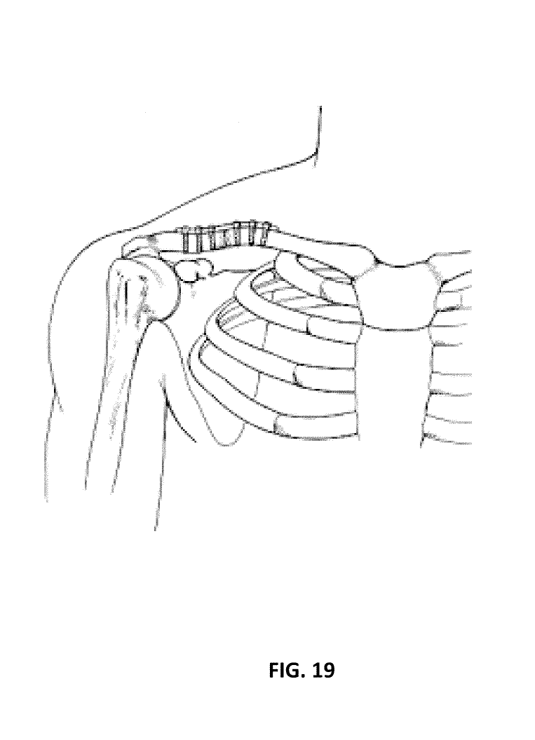

FIG. 20 is an illustration of a patient's skull implanted with smaller size multiple magnet-containing skull screws.

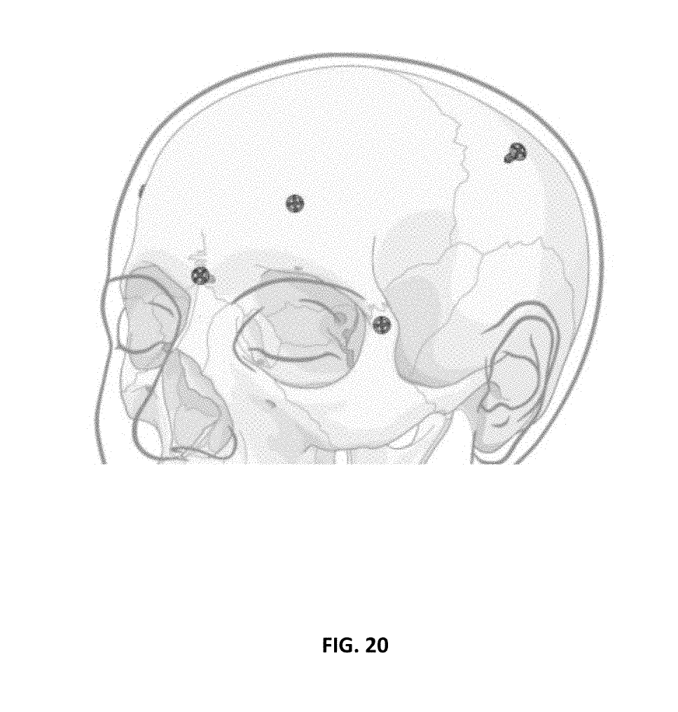

### DETAILED DESCRIPTION OF THE PREFERRED EMBODIMENTS

### Detailed Description of the Invention

Embodiments of the present disclosure will now be described more fully hereinafter with reference to the accompanying drawings, in which some, but not all embodiments of the disclosure are shown.

According to a first aspect, the current disclosure describes an implantable magnetic assembly that includes a rod-shaped magnet housed within a magnetic screw. The magnetic screw has two ends, a point end and a head end, and is configured to traverse a patient's fractured bone, skeletal bone and spinal vertebra with the point end located at the interior surface, towards the affected areas, with the head end located at the exterior surface of the screw. In an alternative embodiment, the magnetic screw is inserted sufficiently deep that the top of the skull screw head end is at the same level as the patient's fractured bone, spinal vertebra or skeletal bone, or between those two levels.

In one embodiment, the magnetic screw may comprise along its length an unthreaded head section and a shaft section, wherein the unthreaded head section is located near the head end, and the shaft section is located near the point end and may be fully or partially threaded.

In one embodiment, the exterior of the magnetic screw may comprise or be made of titanium, titanium alloy, stainless steel, cobalt alloy, carbon fiber, polyethylene, polymethylmethacrylate, polyether ether ketone, polycarbonate, and/or some other biocompatible material. Preferably, the magnetic screw may be comprised of a titanium alloy. To increase biocompatibility, the exterior of the magnetic screw may be anodized or texturized. Alternatively, an ion beam of calcium, potassium, hydroxyapatite, magnesium, nitrogen, and/or argon ions may be used to deposit or implant those ions on the exterior of the magnetic screw. Similarly, the threads and/or exterior surface of the screw may be coated with a material to reduce friction, such as polytetrafluoroethylene (PTFE) and/or ultra-high-molecular-weight polyethylene (UHMWPE). In one embodiment, the magnetic screw comprises materials that can withstand sterilization by autoclaving.

The thread may be single or double start and may be right-handed or left-handed. The design of the screw thread may allow for an implanted magnetic screw to be self-locking, self-tapping, and/or self-drilling. In a preferred embodiment, the magnetic screw may have a lower pullout strength compared to a conventional bone and/or magnetic screw. The shape of the screw threads may be V, American National, British Standard, buttress, Unified Thread Standard, ISO metric, or some different shape known to those of ordinary skill.

The point end of the magnetic screw may be pointed, flat, curved, beveled, or some other shape.

In one embodiment, the magnetic screw shaft section may comprise at least two threaded sections separated by an unthreaded section. The at least two threaded sections may have the same dimensions or they may differ in length, thread diameter, and/or core diameter. In one embodiment, at least two threaded sections are adjacent on the shaft section and differ by thread diameter, and/or core diameter. Alternatively, the shaft section may comprise at least two unthreaded sections separated by a threaded section.

Where a magnetic screw comprises an unthreaded head section, the widest diameter of the unthreaded head section may be greater than the core diameter of the screw. In alternative embodiments, the widest diameter of the unthreaded head section may be equal to or less than the core diameter.

The unthreaded head section of the magnetic screw may be shaped cylindrically, or may be a right prism with a regular polygonal base such as a square, a hexagon, or octagon, or it may be some other shape, such as a sphere or hemisphere.

In one embodiment, the screw has a hollow core in which to receive a rod-shaped magnet. This hollow core may start from the head end of the magnetic screw and terminate before the point end of the magnetic screw.

Preferably, the diameter of the hollow core is nearly the diameter of a cylindrical rod-shaped magnet, but just large enough to allow air to escape when inserting the magnet. Alternatively, a groove or channel may be machined into a side of the hollow core lengthwise to allow air to escape. Alternatively, the hollow core may be a different shape than the rod-shaped magnet.

In one alternative embodiment, the screw is cannulated, meaning that the length of the hollow core equals the length of the screw. In another alternative embodiment, the hollow core starts from the point end of the screw and ends before the head end.

As used herein, “rod-shaped” refers to a right prism or cylinder shape wherein the height of the prism or cylinder is larger than its largest width. In one embodiment, the rod-shaped magnet is a prism with a cylindrical shape, wherein a cylindrical rod-shaped magnet has a height greater than its diameter.

In one embodiment, the rod-shaped magnet is inserted into and then permanently sealed within the magnetic screw, such as by welding or an adhesive. In another embodiment, the rod-shaped magnet is inserted and then secured in place by an adhesive, a solder, a plug, or a cap. The plug or cap may be secured into the head end of the magnetic screw by adhesive or solder or may be fastened by a fastening mechanism such as screw threads or a latch. The fastening mechanism may be on the outside surface of the magnetic screw or within the magnetic screw. In one embodiment, the magnetic screw is made of a ferromagnetic material, and the rod-shaped magnet is secured by magnetic attraction. In one embodiment, the magnetic screw may remain in a patient's fractured bone, spinal vertebra or skeletal bone while the rod-shaped magnet is removed and replaced with a different rod-shaped magnet.

In one embodiment, the magnetic screw may be shaped after inserting the rod-shaped magnet. The rod-shaped magnet may then be inserted and enclosed by welding for example, and the magnetic screw may be machined to a final shape. The magnet may be inserted from the head end or point end of the screw blank, and the magnet insertion and/or welding may be carried out under an inert gas environment. A leak test may be performed to ensure that the magnet has been completely sealed within the magnetic screw.

In an alternative embodiment, the magnetic screw is not machined to create a hollow core, but cast with the rod-shaped magnet embedded within the screw.

The head end of the screw may comprise a screw driver such as a Phillips, a slot, a hex, a hex socket, a Torx, a double square, a square, a Robertson, or some other screw drive.

In another embodiment, the magnetic screw may contain more than one rod-shaped magnet, and these magnets may be arranged with at least one rod-shaped magnet having a north pole pointing towards the head end of the screw and at least one other rod-shaped magnet having a south pole pointing towards the head end of the screw in order to create a multipolar magnetic field with steep magnetic field gradients.

In an alternative embodiment, the interior of the magnetic screw may be filled with a stack of flat magnets. In another alternative embodiment, the magnetic screw may contain a magnet or magnets with magnetic poles antiparallel to the length of the magnetic screw. In another alternative embodiment, the skull screw may contain magnets of different diameters and/or lengths so that the shape and strength of the combined magnetic field may be customized.

According again to the first aspect, the implantable magnet assembly also comprises a casing removably attached to the head end of the magnetic screw, with the casing housing at least one flat magnet, and the at least one flat magnet is arranged substantially perpendicularly with the rod-shaped magnet. As used herein, “flat” refers to a right prism or cylinder shape wherein the largest width of the prism or cylinder is greater than its height. In one embodiment, this flat shape is a disc, meaning a cylindrical flat magnet that has a diameter greater than its height.

In one embodiment, the magnet or magnets in the casing and/or skull screw may comprise ferrite (also known as ceramic magnets, such as BaFe₂O₃ and SrFe₂O₃), neodymium (such as Nd₂Fe₁₄B), samarium-cobalt (such as SmCo₅ or Sm₂Co₁₇), alnico, or any combination thereof. The magnet or magnets in the casing may comprise different material than the magnet or magnets in the magnetic screw, but preferably, they comprise the same material, and in a preferred embodiment, the magnets comprise neodymium. The magnets may be plated with nickel or coated with another substance to prevent corrosion.

In one embodiment, the combined magnetic strength of the magnets within the implantable magnet assembly results in a magnetic field strength of 2 mT-15 T, preferably 4 mT-10 T, more preferably 5 mT-5 T as measured from any exterior surface of the implantable magnet assembly. In an alternative embodiment, electromagnets may be used instead of permanent magnets to produce similar magnetic field strengths. In a further embodiment, electromagnets may be used to deliver pulsed magnetic fields.

In one embodiment, the magnet or magnets in the casing have a flat shape, as defined earlier, and may be rectangular prisms, discs, triangular prisms, hexagonal prisms, octagonal prisms, elliptic cylinders, or some other flat shape.

In an alternative embodiment, the magnets in the casing are not flat magnets, but some other shape or shapes, such as prismoids, spheres, toroids, polyhedra, or irregularly-shaped solids. In one alternative embodiment, the magnets in the casing are flat rings of varying inner and outer diameters that allow them to be arranged concentrically with the magnetic screw and with each other. In another alternative embodiment, rather than being strictly rod-shaped or disc-shaped, the magnet in the skull screw and/or the magnet or magnets in the casing are of a right prism or cylinder shape each with a height equal to its largest width.

Preferably, the flat magnet or flat magnets may be arranged substantially perpendicularly with the rod-shaped magnet. As defined here, “arranged substantially perpendicularly” means that an axis along the length of the rod-shaped magnet intersects at an angle 70-110°, preferably 75-110°, more preferably 80-100° with the geometric planes that contain the two largest faces of the flat magnet, where 90° is a true perpendicular angle. In this arrangement, an extension of an axis along the length of the rod-shaped magnet may or may not intersect with a flat magnet. Furthermore, the rod-shaped magnet may or may not touch a flat magnet. In the case where the rod-shaped magnet does not touch a flat magnet, preferably the shortest distance between them is less than 20 mm, preferably less than 10 mm, more preferably less than 5 mm. Alternatively, in one embodiment, the flat magnet has a hole that accommodates a part of a rod-shaped magnet protruding from the head end of the magnet screw and through the casing.

In one embodiment, the implantable magnet assembly has one flat magnet which is cylindrically-shaped and aligned concentrically with the rod-shaped magnet with like magnetic poles of both magnets facing the same direction. By having the magnetic poles of the two magnets facing the same direction, with or without the magnets touching each other, the strength of the magnetic field may be combined and increased, and may penetrate deeper into the brain of a patient.

In one embodiment, the implantable magnet assembly has at least two flat magnets with edges arranged side-by-side in the casing. The magnets may touch each other, or they may be separated from their nearest neighbor by at most 10 mm, preferably at most 5 mm, more preferably at most 1 mm.

Where two or more flat magnets are housed in the casing, the flat magnets may not be arranged substantially perpendicularly with the rod-shaped magnet, but preferably at least one flat magnet is. In an alternative embodiment, two or more flat magnets housed in the casing may not share the same geometric plane due to the casing being shaped to the curve of a patient's head.

Where two or more flat magnets are housed in the casing, preferably the flat magnets are the same shape, size, and composition, or they may be different. For instance, to create an asymmetric magnetic field, one flat magnet may cover a larger area and/or create a stronger magnetic field. In an alternative embodiment, two or more flat magnets may be stacked within the casing.

In a further embodiment, where the implantable magnet assembly has at least two flat magnets with edges arranged side-by-side in the casing, not every flat magnet is arranged with magnetic poles facing in the same direction.

In another embodiment, where the implantable magnet assembly has at least two flat magnets with edges arranged side-by-side in the casing, all of the flat magnets are arranged with their magnetic poles in the same direction as the magnetic pole of the rod-shaped magnet.

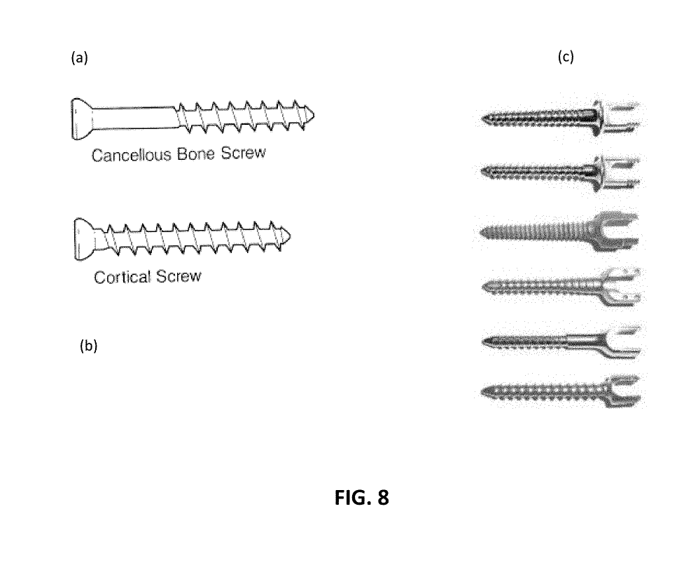

In one embodiment, the casing may be a flat shape that approximates the shape of the flat magnet or flat magnets contained inside. For example, a disc-shaped magnet may be housed in a disc shaped casing, and two disc-shaped magnets with edges side-by-side may be housed in a FIG. 8 shaped casing.

In one embodiment, the casing and the magnetic screw may both comprise the same material, such as a titanium alloy. In another embodiment, the casing may comprise a different material than the magnetic screw. The casing may be any of the previously mentioned materials suitable for the magnetic screw, or the casing may be another metal such as nickel and/or aluminum, or another polymeric material such as polylactic acid (PLA), poly(lactic-co-glycolic acid) (PLGA), polyethylene terephthalate (PET), acrylonitrile butadiene styrene (ABS), and/or polytetrafluoroethylene (PTFE), or some other non-metal, such as glass or ceramic. Preferably, the magnetic screw and the casing are made of the same material. In an alternative embodiment, no casing is present and a flat magnet attaches directly to the head end of the magnetic screw.

In another embodiment, the casing may be a flat shape but does not approximate the shape of the flat magnet or flat magnets contained inside.

In one embodiment, the casing may include a cushion on a part of the casing that contacts a patient's bone. The part of the casing in contact with a patient's bone may entirely comprise a cushion, or only a portion may comprise a cushion, for example, in the form of raised ridges or bumps. Alternatively, the entire exterior surface of the casing, including surfaces that are not in contact with a patient's bone, may comprise a cushion. The cushion may comprise an elastomeric compound such as those previously discussed, and may be solid or further comprise air pockets.

In one embodiment, the casing and the head end of the magnetic screw have a fastening mechanism to removably attach the two. Preferably, the casing can be attached or detached from a magnetic screw implanted in a patient's bone without changing the positioning of the magnetic screw and without causing discomfort to the patient. In one embodiment, this fastening mechanism may be a bayonet mount, a threaded connector, a clutch, a latch, a key and keyhole, a tongue and groove joint, a snap fastener, an R-clip, a clamp, or any combination thereof. The fastening mechanism may comprise additional parts such as pins, springs, tabs, or levers. The fastening mechanism may reside on the exterior side of the screw near the head end, on the top of the head end of the magnetic screw, within a recession in the head end of the magnetic screw, or any combination thereof, with the complementary fastening mechanism located on an exterior surface projecting from the casing, on an interior surface of an indentation of the casing, on an exterior surface flush with the casing, or any combination thereof.

In an alternative embodiment, the magnetic attraction between the magnet in the magnetic screw and a magnet in the casing may be sufficient to removably attach the casing to the head end of the skull screw. In another alternative embodiment, a plug is used to seal the rod-shaped magnet in the magnetic screw, as mentioned previously, and this plug may comprise a fastening mechanism to removably attach the casing.

In a related embodiment, the rod-shaped magnet is not sealed inside the magnetic screw with a plug, but is secured by direct contact with a flat magnet housed in the casing.

In another embodiment, the exterior surface of the casing comprises a tab portion, a notch, or a textured surface to facilitate a finger grip. A tab may be in the form of a projection on the surface of the casing while a notch may be in the form of a V-shaped cut. The exterior surface of the casing may be textured with grooves, bumps, knurls, ridges, and/or ribs. A tab, notch, or textured feature may be present in any combination or number on any exterior surface of the casing not in contact with a patient's bone. In one embodiment, to facilitate a finger grip, the casing is covered with a cushion of an elastomeric material, such as those listed previously.

In one embodiment, the magnetic screw and a bottom portion of the casing are machined from a single piece of material. To provide access to the flat magnet or both flat and rod-shaped magnets, a top portion of the casing may be removably attached to the bottom portion.

In an alternative embodiment, the entire casing may be permanently attached to the head end of the magnetic screw, and the implantable magnet assembly may be implanted and taken out of a patient's bone as one single piece.

The magnets may be secured within the casing by an adhesive, or by a structure inside the casing. This structure may be unmovable, such as a slot or depression, or it may be moveable, such as a clamp or clip. In one embodiment, the casing or a portion of the casing is made of a ferromagnetic material to which the magnets are attracted. In one embodiment, the casing has a top portion removably attached to a bottom portion, and the attachment of the top portion to the bottom portion is sufficient to secure the magnet. In this embodiment, the casing may be opened in order to exchange the magnets. In a further embodiment, the casing may be opened while leaving a bottom portion of the casing still attached to the magnetic screw.

In one embodiment, the casing may not be entirely flat, but curved to the shape of a person's bone. It is possible that once a magnetic screw location has been determined, a customized casing may be fabricated to better fit the local curve of a patient's bone.

According to a second aspect, the present disclosure relates to a method of delivering a static magnetic field to a patient's bone by implanting the implantable magnet assembly, in one or more of its embodiments, into the patient's fractured bone, spinal vertebra or skeletal bone where the magnetic screw traverses the affected area with the point end located at the interior surface and the head end located at the exterior surface. The magnetic field may produce neuronal excitatory and/or inhibitory effects by changing the polarity of different neurons. The reverse polarity of the magnetic field may or may not produce the same effects on a patient's bone. Different magnetic implant locations, as well as the shape, strength, and polarity of the magnetic fields, may lead to different treatment options. In one embodiment, a patient's bone may first be imaged by MRI, positron emission tomography (PET) and/or computed tomography (CT) to better determine these parameters.

The implanting step may require surgery under anesthesia, and may involve shaving a portion of the patient's head and cutting a segment of the scalp to expose an exterior portion of the skull. Depending on the design of the magnetic screw, a hole or incision in the patient's bone may be required. In one embodiment, the entire implantable magnet assembly may be inserted as one piece into a patient's bone.

In a preferred embodiment, where the implantable magnet assembly comprises a casing removably attached to the magnetic screw, the screw may be implanted first, and then the casing may be attached. Alternatively, the magnetic screw may be inserted first, and then after a period of healing for the fractured bone, spinal vertebra or skeletal bone, the casing may be attached. A surgeon may use a powered orthopedic screwdriver to insert the magnetic screw or implantable magnet assembly, or may use non-powered surgical hand tools. Preferably, the magnetic screw is inserted substantially perpendicularly to the affected area. In a preferred embodiment the magnetic screw is not implanted to a depth that would cause hard to the bone tissue.

In a related embodiment, the diameter of the magnetic screw unthreaded head section and length of the shaft section are chosen so that the magnetic screw point end cannot penetrate far.

In an alternative embodiment, the magnetic screw may be inserted sufficiently deep to anchor to the affected area without puncturing the interior surface of the bone. In another alternative embodiment, instead of implanting the assembly directly into the bone, a receptacle for the screw is implanted into the affected area, and the implantable magnet assembly is inserted into the receptacle. In another alternative embodiment, a hole is drilled into a patient's bone and an unthreaded magnetic screw is inserted and secured by osseointegration with the bone tissue. In another alternative embodiment, where the rod-shaped magnet is inserted after implanting the magnetic screw, the hollow core of the magnetic screw may be used as a screw drive.

In one embodiment of the method, the static magnetic field is delivered for more than 2 hours. Preferably the static magnetic field may be delivered for more than one week, preferably more than four weeks. To deliver the static magnetic field for a certain amount of time, the implantable magnet assembly is left in place for that time period. Given the secure design of the implantable magnet assembly, the delivery of the static magnetic field may occur on an outpatient basis, meaning that a patient may not require constant medical supervision nor have to significantly restrain his or her lifestyle. Alternatively, types of psychological or medical evaluation may be performed during the time period of magnetic field delivery in order to detail the effects of the treatment. These evaluations may be questionnaires, blood tests, vital signs, electroencephalography (EEG), electrocardiography (ECG/EKG), or imaging. In addition, other therapies may coincide with the period of magnetic stimulation, such as chemotherapy, psychological counseling.

In a further embodiment of the method, the method additionally involves removing the casing while leaving the rod-shaped magnet and magnetic screw in place and attaching a second casing with at least one second flat magnet, wherein the at least one second flat magnet applies a magnetic field that differs from the magnetic field produced by the at least one flat magnet. Evaluations of a patient with the implantable magnet assembly may warrant changes to the magnetic field strength or shape. This may be accomplished by exchanging the casing for a second casing that may deliver a different static magnetic field. In one embodiment, the casing may be exchanged by a physician without using specialized tools. In a related alternative embodiment, mentioned previously, a top portion of the casing may be opened in order to exchange, remove, and/or add magnets without separating the entire casing from the implanted magnetic screw.

Following the required course of treatment, the magnets or the entire implantable assembly may be removed from the patient. In an alternative embodiment, more than one magnet assembly may be implanted into a patient's bone.

These assemblies may be implanted or removed at the same medical appointment, or at different appointments. The more than one magnet assembly may each produce magnetic fields of the same shape and strength, or they may produce different magnetic fields. For implantable magnetic assemblies on directly opposing sides of a patient's bone, the magnetic polarities of all the magnets may be aligned in one direction, for example, the north poles of the magnets may all point towards the left side of the patient. Such an arrangement may create a unipolar magnetic field through the center of the patient's brain. Alternatively, implantable magnetic assemblies on opposing sides of a patient's head may contain magnets where the poles are not all aligned in a single direction.

In one embodiment of the method, a patient has a osteoporosis/or a neurological ailment, and the condition the is treated by the static magnetic field from the implantable magnet assembly. As used herein, to “treat” osteoporosis or a neurological ailment means to reduce or inhibit the progression, severity, and/or duration of the condition or other accompanying symptoms. As mentioned previously, another form of therapy may be combined with the magnetic stimulation.

In one embodiment, the magnetic screw and the casing contain no magnets, in order to serve as a placebo for magnetic stimulation. In this embodiment, the magnetic screw and the casing may contain rod-shaped and flat-shaped pieces of material, such as a non-magnetic metal, in order to approximate the shape and density of the magnets being replaced. With this type of placebo assembly, a patient's bone may be implanted with the placebo and evaluated. Alternatively, the patient with the placebo may be compared to a patient or patients with a magnet-containing assembly of similar appearance. Alternatively, a patient's bone may be implanted with two or more assemblies, with at least one containing magnets and at least one as a magnet-free placebo.

In a related embodiment of the implantable magnet assembly, the magnetic screw may contain no magnets, while the casing contains at least one flat magnet.

According to a third aspect, the present disclosure relates to a method of treating a patient's fractured bone, spinal vertebra or skeletal bone with a static magnetic field by implanting the magnetic screw containing the rod-shaped magnet, but not attaching the casing or flat magnets.

In one embodiment of this method, a patient may have osteoporosis/or a neurological ailment, the result is that it is treated. The ailment may be any one or a combination of those previously listed, and the methods of treatment are similar to what was mentioned previously for the implantable magnet assembly comprising at least one flat magnet.

The examples below are intended to further illustrate the construction of the implantable magnet assembly and methods of use and are not intended to limit the scope of the claims.

Embodiments of the present disclosure will now be described more fully hereinafter with reference to the accompanying drawings, in which some, but not all embodiments of the disclosure are shown.

1: An implantable magnet assembly comprising:
a rod-shaped magnet housed within a magnetic screw, wherein the screw has a head end and a point end and is configured to traverse a patient's bone with the point end located at the interior and the head end located at the exterior surface; a casing removably attached to the head end of the magentic screw; and at least one flat magnet housed in the casing; wherein the rod-shaped magnet and the flat magnet are arranged substantially perpendicularly.

2: The implantable magnet assembly of claim 1 which emits a magnetic field with a magnetic field strength of 2 mT-15 T as measured on an exterior surface of the implantable magnet assembly.

3: The implantable magnet assembly of claim 1 which has one flat magnet which is cylindrically-shaped and aligned concentrically with the rod-shaped magnet with like magnetic poles facing the same direction.

4: The implantable magnet assembly of claim 1 which has at least two flat magnets with edges arranged side-by-side in the casing.

5: The implantable magnet assembly of claim 4 wherein not every flat magnet is arranged with magnetic poles facing in the same direction.

6: The implantable magnet assembly of claim 4 wherein the at least two flat magnets are arranged with magnetic poles in the same direction, and the direction of the poles of the at least two flat magnets are arranged in the same direction as a magnetic pole of the rod-shaped magnet.

7: The implantable magnet assembly of claim 1 wherein the exterior of the magnetic screw comprises at least one selected from the group consisting of titanium, titanium alloy, stainless steel, cobalt alloy, carbon fiber, polyethylene, polymethylmethacrylate, polyether ether ketone, and polycarbonate.

8: The implantable magnet assembly of claim 1 wherein the exterior surface of the casing comprises a tab portion, a notch, or a textured surface to facilitate a finger grip.

9: The implantable magnet assembly of claim 1 wherein the casing is removably attached to the head end of the magnetic screw by a fastening mechanism selected from the group consisting of a bayonet mount, a threaded connector, a clutch, a latch, a key and keyhole, a tongue and groove joint, a snap fastener, an R-clip, and a clamp.

10: The implantable magnet assembly of claim 1 further comprising a strap with a recess to receive the casing, wherein the strap is configured to secure the casing in place when encircling a part of a skeletal bone of the patient.

11: The implantable magnet assembly of claim 1 further comprising a cushion disposed on a bottom portion of the casing that receives the head end of the magnetic screw to provide a cushion to the affected area.

12: The implantable magnet assembly of claim 1 wherein a central portion of the casing on a side of the casing closest to the patient's bone comprises an indentation to receive the head end of the magnetic screw.

13: A method of delivering a static magnetic field to a bone of a patient comprising: implanting the implantable magnet assembly of claim 1 into the patient's bone, wherein the bone screw traverses the patient's bone with the point end located at the interior surface of the bone and the head end located at the exterior surface of the bone.

14: The method of claim 13 wherein the static magnetic field is delivered for more than 2 hours.

15: The method of claim 13 wherein a cognitive performance of the patient is increased relative to a second patient not receiving a magnetic field from a magnet or an electromagnet.

16: The method of claim 13 wherein the patient has a an orthopedic ailment, and it is treated.

17: The method of claim 16, wherein the patient has at least one orthopedic ailment selected from a group of diseases

18: The method of claim 13 further comprising: removing the casing while leaving the rod-shaped magnet and skull screw in place; attaching a second casing comprising at least one second flat magnet, wherein the at least one second flat magnet applies a second magnetic field that differs from a magnetic field produced by the at least one flat magnet.

19: A method of delivering a static magnetic field to a skeletal bone of a patient comprising: implanting an implantable magnet into the patient's bone, wherein the implantable magnet comprises a rod-shaped magnet housed within a magnetic screw comprising a head end and a point end and wherein the implantable magnet is implanted with the point end of the magnetic screw located at the interior surface of the bone and the head end located at the exterior surface of the bone

20: The method of claim 19 wherein the patient has a orthopedic ailment, and it is treated.
